(19) United States
(12) Patent Application Publication
(10) Pub. No.: US 2024/0058427 A1
(43) Pub. Date: Feb. 22, 2024

(54) AVIAN FOOD ADDITIVE

(71) Applicant: PEPSIS LIMITED, London (GB)

(72) Inventor: Rosemary Waring, London (GB)

(73) Assignee: PEPSIS LIMITED, London (GB)

(21) Appl. No.: 18/269,663

(22) PCT Filed: Dec. 24, 2021
(86) PCT No.: PCT/GB2021/053435

§ 371 Date: Jun. 26, 2023

(30) Foreign Application Priority Data
Dec. 24, 2020 (GB) 2020617.3

(51) Int. Cl. A61K 38/47; A23K 10/30; A23K 20/189; A23K 20/163; A23K 20/158; A23K 20/105; A23K 50/70; A23K 50/75; A61K 9/00; A61K 36/899; A61K 38/46; A61K 38/48; A61P 31/04

(52) U.S. Cl. CPC A61K 38/47; A23K 10/30; A23K 20/189; A23K 20/163; A23K 20/158; A23K 20/105; A23K 50/70; A23K 50/75; A61K 9/0056; A61K 36/899; A61K 38/465; A61K 38/48; A61P 31/04

(57) ABSTRACT

This invention relates to a feed additive for use in, amongst other things, improving energy utilisation originating from feed for an avian granivore, the feed additive comprising a malt extract comprising one or more enzymes selected from the group consisting of amylases, maltases, cellulases, fructanases, glucanases, xylanases and deacetylases.

This invention relates to a feed additive for use in, amongst other things, improving energy utilisation originating from feed for an avian granivore, the feed additive comprising a malt extract comprising one or more enzymes selected from the group consisting of amylases, maltases, cellulases, fructanases, glucanases, xylanases and deacetylases.

Racing pigeons are bred from domestic pigeons (Columba livia domestica) and trained to return home (‘race’) from a start point, which may be many miles distant, flying at speeds of 50-60 miles per hour (mph). Consequently, the birds arrive exhausted, often carrying infections picked up from other birds released at the same time. These infections can be transmitted to other birds in their lofts and hence the mortality of young birds from infections is greater than 30%, even with experienced owners who maintain good levels of hygiene.

The level of stress amongst racing pigeons is also high, both from racing itself and from the carriage conditions to the release points before the race. This results in microbiome changes and bowel dysfunction with loose, watery or green droppings.

There are a number of factors which play a role in successfully racing pigeons. Infection from parasites, bacteria (especially Escherichia coli, Staphylococcus and Salmonella), viruses and the fungus Candida all reduce performance and must therefore be controlled. Optimising both the performance itself (race time), the speed of recovery from a race, and the capacity to repeat-race quickly are also essential components in success. Typically, pigeons race on Saturday and/or Sunday. They are then allowed to recover on Monday before re-starting feeding and training for the next weekend. As prize money in pigeon racing is relatively modest, most owners race their birds as frequently as possible during the racing season which lasts from March to August.

Owners use a wide range of feed in attempts to improve the performance of their birds. According to Grond et al. (‘Longitudinal microbiome profiling reveals impermanence of probiotic bacteria in domestic pigeons’, PLoS ONE 14(6): e0217804 (2019)), probiotics are commonly used by pigeon owners. However, probiotic shifts in the microbiome composition have been shown to be temporary, disappearing within two days of cessation of treatment with Lactobacillus acidophilus pellets whist administration of the probiotic in drinking water had no effect.

According to Abd El-Khalek et al. (‘Indirect evidence for microbiota reduction through dietary mannanoligosaccharides in the pigeon’, J. Animal Physiol. and Animal Nutrition, 96(6), 1084-90 (2012)), diet components can affect the gut microbiome. Thus in pigeons, dietary mannano-oligosaccharides acidified the droppings by increasing excretion of uric acid which appeared to reduce the gut bacterial challenge.

Amann et al. (‘Exocrine pancreatic insufficiency in pigeons’, Avian Path. 35(1), 58-62 (2006)) describes that the exocrine pancreatic enzymes found in avian duodenum are amylase, lipase, trypsin and chymotrypsin, whose function is to digest carbohydrates.

The feed conversion ratio (FCR, kg weight of feed/kg weight gain) in pigeons is relatively low increasing, according to Darwati et al. (J. Indonesian Trop. Anim. Agric., 35, 4, 268 (2010)), from 2.95 to 10.16 from the first week after hatching to the fourth week. In comparison, the FCR in pheasants is 4.5 (NSW Government, Department of Primary Industries, Animals and Livestock, Poultry and Birds, Poultry Species, Feeding Pheasants, Food Consumption) and, according to Best (‘Poultry performance improves over past decades’, WATTAgNet.com, 24 Nov. 2011 (https://web.archive.org/web/20160616092918/http://www.wattagnet.com/articles/10427-poultry-performance-improves-over-past-decades)), in chickens is 1.6-2.0. Xu et al. (‘Effects of dietary fructooligosaccharides on digestive enzyme activities, intestinal microflora and morphology of male broilers’, Poultry Science, 82, 1030-1036 (2003)) discloses, however, that even the efficient chicken can benefit from improvements in their microbiome since dietary fructooligosaccharides (FOS) at a rate of 4.0 g/kg feed significantly increased average daily weight gain in broilers, enhancing growth of Bifidobacterium and Lactobacillus, and inhibited E. coli in the small intestine and caecum.

Chickens (Gallus gallus domesticus) are commercially very important. Desirable characteristics in laying birds are increases in weight over the laying period, even after the metabolic drain of egg production, and improvements in egg quality. In commercial operations, egg losses are largely due to weak eggshells that crack on handling and/or transport. Morbidity in chickens also leads to economic loss where one of the major factors is footpad dermatitis due to the cage litter being permanently damp from wet droppings (Abraham et al., ‘Orange corn diets associated with lower severity of footpad dermatitis in broilers’, Poultry Science, 100, 5, 101054 (2021)).

### SUMMARY OF THE INVENTION

The inventors observed, amongst other things, an improvement in racing recovery and performance in racing pigeons fed a barley-based malt extract attributed to improvements in the gut microbiome.

In a first aspect of the invention, a feed additive for use in treating and/or preventing one or more pathogenic bacteria in an avian granivore is provided, the feed additive comprising a malt extract comprising one or more enzymes selected from the group consisting of amylases, maltases, cellulases, fructanases, glucanases, xylanases and deacetylases.

In a second aspect of the invention, a feed additive for use in improving treatment and/or prevention by an effective amount of one or more antibiotics of one or more pathogenic bacteria in an avian granivore is provided, the feed additive comprising a malt extract comprising one or more enzymes selected from the group consisting of amylases, maltases, cellulases, fructanases, glucanases, xylanases and deacetylases.

In a third aspect of the invention, a feed additive for use in reducing the incidence of viral infections in an avian granivore is provided, the feed additive comprising a malt extract comprising one or more enzymes selected from the group consisting of amylases, maltases, cellulases, fructanases, glucanases, xylanases and deacetylases.

In a fourth aspect of the invention, a feed additive for use in reducing post-race recovery time for Columba livia domestica is provided, the feed additive comprising a malt extract comprising one or more enzymes selected from the group consisting of amylases, maltases, cellulases, fructanases, glucanases, xylanases and deacetylases.

In a fifth aspect of the invention, a feed additive for use in improving energy utilisation originating from feed for an avian granivore is provided, the feed additive comprising a malt extract comprising one or more enzymes selected from the group consisting of amylases, maltases, cellulases, fructanases, glucanases, xylanases and deacetylases.

In a sixth aspect of the invention a feed additive for use in improving voluntary feed intake of a granivore is provided, the feed additive comprising a malt extract comprising one or more enzymes selected from the group consisting of amylases, maltases, cellulases, fructanases, glucanases, xylanases and deacetylases.

In a seventh aspect of the invention a feed additive for use in increasing egg weight of an avian granivore is provided, the feed additive comprising a malt extract comprising one or more enzymes selected from the group consisting of amylases, maltases, cellulases, fructanases, glucanases, xylanases and deacetylases.

In an eighth aspect of the invention, a feed additive for use in increasing eggshell thickness of an avian granivore is provided, the feed additive comprising a malt extract comprising one or more enzymes selected from the group consisting of amylases, maltases, cellulases, fructanases, glucanases, xylanases and deacetylases.

In a ninth aspect of the invention, a feed additive for use in improving the feed conversion ratio of an avian granivore is provided, the feed additive comprising a malt extract comprising one or more enzymes selected from the group consisting of amylases, maltases, cellulases, fructanases, glucanases, xylanases and deacetylases.

In a tenth aspect of the invention, a feed additive for use in increasing the firmness of avian granivore droppings is provided, the feed additive comprising a malt extract comprising one or more enzymes selected from the group consisting of amylases, maltases, cellulases, fructanases, glucanases, xylanases and deacetylases.

### SUMMARY OF THE FIGURES

The invention is described in more detail below with reference to:

FIG. 1 which shows the average percentage hits of Lactobacillus, Ruminococrus, Lachnospiracea, and Bifidobacterium for five egg laying chickens (denoted 1 to 5) before (‘begin’) and after (‘end’) two months of treatment with EquiNectar (denoted ‘C’) versus five egg laying control chickens where water is substituted for Equinectar (denoted ‘A’); and

FIG. 2 which shows the average percentage hits of Enterobacteriales, Clostridiales, and Campylobacterales for five egg laying chickens (denoted 1 to 5) before (‘begin’) and after (‘end’) two months of treatment with EquiNectar (denoted ‘C’) versus five egg laying control chickens where the water is substituted for Equinectar (denoted ‘A’).

### DETAILED DESCRIPTION OF THE INVENTION

In a first aspect of the invention, a feed additive for use in treating and/or preventing one or more pathogenic bacteria in an avian granivore is provided, the feed additive comprising a malt extract comprising one or more enzymes selected from the group consisting of amylases, maltases, cellulases, fructanases, glucanases, xylanases and deacetylases.

In a second aspect of the invention, a feed additive for use in improving treatment and/or prevention by an effective amount of one or more antibiotics of one or more pathogenic bacteria in an avian granivore is provided, the feed additive comprising a malt extract comprising one or more enzymes selected from the group consisting of amylases, maltases, cellulases, fructanases, glucanases, xylanases and deacetylases.

Preferably the one or more pathogenic bacteria are gastrointestinal pathogenic bacteria, more preferably the one or more pathogenic bacteria is selected from the group consisting of Escherichia coli, Salmonella enteritidis, Campylobacter spp, and Staphylococcus spp.

The malt extract preferably additionally comprises one or more proteinases and/or lipases.

The malt extract preferably additionally comprises one or more water soluble sugars selected from the group consisting of maltose, maltotriose, and maltose polymers.

The daily dosage of the malt extract is preferably 0.3-30, 0.4-20, 0.5-10, 0.7-5, 1-3 g per 1000 g granivore body weight.

EquiNectar (Tharos Limited, London, England) is an animal feed additive comprising a barley malt extract, coconut oil (a medium chain triglyceride (MCT) comprising medium chain fatty acids (C₆-C₁₂) (MCFAs)), and the preservative potassium sorbate (sorbic acid is found in many plants including rowan berries).

According to Peh et al. (‘Antimicrobial activity of organic acids against Campylobacter spp. and development of combinations—A synergistic effect?’, PLoS ONE [Electronic Resource], 15, 9, :e02393122020), sorbic acid is active against Campylobacter spp. which are major contaminants of poultry meat and reduce avian health.

The barley malt extract itself comprises a plurality of enzymatically active digestive enzymes, in particular alpha- and/or beta-amylase (alpha-amylase breaks starch down yielding maltotriose and maltose from amylose, and maltose, glucose and limit dextrin from amylopectin and beta-amylase breaks down starch into maltose), maltase, cellulase, fructanases (which break down fructans found in grass), glucanases (which break down glucans found in cell walls), xylanases (which break down xylans in plant cell walls), deacetylases (which cleave acetyl groups from xylans and fructans thereby allowing xylanases and fructanases to break down the remainder of the molecular structure), and smaller amounts of proteinases and lipases (which break down respectively proteins and fats). The malt extract also comprises maltose, maltotriose and maltose polymers, and, depending on the precise parameters used in the process of preparing the malt extract, peptides and/or amino acids. The malt extract does not, however, comprise starch because the starch in the barley seeds is broken down to produce a mixture of the aforementioned maltose, maltotriose and maltose polymers.

The malt extract is prepared by soaking barley seeds in water in order to germinate the seeds. Germination causes the seeds to produce a variety of enzymes that break down, for example, starches into sugars through the production of amylases and other carbohydrases, such as fructanases. The germination process also induces other enzymes such as proteases that break down proteins in the grain. Germination is halted by drying with hot air at a temperature of no higher than about 75, 70, 65, 60, 55, 50, 45 or 40 degrees centigrade thereby to produce a malt. Whilst higher temperatures may be used to dry the germinated seeds, such higher temperatures denature an ever greater proportion of the enzymes present in the malt.

The dried sprouted seeds are then milled and water is added and heated to at least about 40, 45, 50, 55, 60, 65 but below 75 or 70 degrees centigrade in order to form a mash, and stirred for about one hour. The enzymes present are active at different temperatures. Thus proteases and beta-amylases are active at about 50 degrees centigrade. Thereafter at about 65 degrees centigrade alpha-amylases degrade starch to sugars. The next step is separation of the residual solids (‘spent grain’) from the liquid (‘wort’). The wort is then concentrated by vacuum evaporation to provide an active enzyme rich malt extract, typically comprising about 80% w/w solids in a solution also rich in sugars.

The malt extract typically has a diastatic power value of above 35 degrees Lintner (94 degrees Windisch Kolbach (WK) units), or above 40, 45, 50, 55, 60, 65, 70, 75 or 80 degrees Lintner. For comparative purposes, a malt with enough power to self-convert starch to sugars has a diastatic power of about 35 degrees Lintner.

Whilst EquiNectar comprises a malt extract based on barley seeds, potentially any seed may be used to produce the malt. For example, wheat, triticale, sorghum, maize, buck wheat or rice may be used.

According to page 3 (lines 8-12 and 30-32) of WO 2018/096334 (Pepsis Limited), the medium chain triglyceride may be useful for improving the digestion of food as well as increasing glycogen availability in muscles and providing additional energy. As pancreatic alpha-amylases are activated by fatty acids, the addition of MCT (which hydrolyses to fatty acids) boosts the activity of the enzymes in the gut. Attia et al. (‘The effects of different oil sources on performance, digestive enzymes, carcass traits, biochemical, immunological, antioxidant and morphometric responses of broiler chicks’, Frontiers in Veterinary Science, 7, 181 (2020)) describes a study showing that coconut oil increases gut amylase activity as well as providing significant increases in blood plasma antibodies immunoglobulin G (IgG) and immunoglobulin M (IgM). It also improves antioxidant status, the antibody titre to avian influenza and the respiratory disease Newcastle disease, and the feed conversion ratio.

Whilst EquiNectar comprises coconut oil, other MCTs may be used including those having two or three different medium chain fatty acids (MCFAs) selected from the group consisting of caproic acid (C₆), caprylic acid (C₈), capric acid (C₁₀) and lauric acid (C₁₂).

Typically the animal feed additive preferably comprises about 1-5, 1-10, 1-15% w/w one or more MCTs. MCTs comprising at least 40 or 45% w/w lauric fatty acid are preferred, such as coconut oil, for the reasons provided hereinabove. Furthermore it has been observed by Hafeez et al. (‘Effect of diet supplemented with coconut essential oil on performance and villus histomorphology in broilers exposed to avian coccidiosis’, Tropical Animal Health and Production, 52, 5, 2499 (2020)) that using 2% coconut oil as a dietary supplement for broiler chicks led to significantly better feed conversion ratios than controls even when challenged with avian coccidiosis. The supplemented chicks also had improved gastrointestinal tract villus histology with higher length, width and surface area. In addition and as reported by Sefi et al. (‘Short chain fatty acids may improve hepatic mitochondrial energy efficiency in heat-stressed broilers’, J. Thermal Biology, 89, 102520 (2020)), as compared with other oils (long-chain saturated fatty acids in the form of beef tallow, monounsaturated fatty acids in the form of olive oil, and polyunsaturated fatty acids in the form of soybean oil), dietary coconut oil improves hepatic mitochondrial energy efficiency in heat-stressed broilers giving the highest adenosine triphosphate (ATP) concentration and mitochondrial membrane potential.

The feed additive preferably additionally comprises an effective amount of one or more water soluble organic acid or salt thereof. The water soluble organic acid is optionally selected from the group consisting of caprylic acid, sorbic acid, caproic acid, benzoic acid, ascorbic acid, propionic acid, acetic acid, formic acid, fumaric acid, and tartaric acid, and is preferably sorbic acid, more preferably a salt of sorbic acid, for example potassium sorbate. EquiNectar comprises about 1% w/w potassium sorbate. Thus the feed additive preferably comprises 0.001-5, 0.01-3, 0.05-2, 0.1-1.5, 0.5-1.25, 0.75-1.2% w/w of one or more water soluble organic acid or salt thereof.

The feed additive is typically used in combination with granivore feed, for example grain. Typical pigeon feed consists of flint maize, wheat, red Dari, plate maize, white Dari, maple peas, safflower seed, and tares (‘Bucktons Super Widowhood’, Bucktons, Driffield, East Yorkshire, UK), and red maize, maple peas, red Dari, plate maize, white Dari, tares, blue peas, white peas, and safflower seed (‘Irish Ruby’, Bucktons, Driffield, East Yorkshire, UK).

The avian granivore is preferably Columba livia domestica or Gallus gallus domestica.

In a third aspect of the invention, a feed additive for use in reducing the incidence of viral infections in an avian granivore is provided, the feed additive comprising a malt extract comprising one or more enzymes selected from the group consisting of amylases, maltases, cellulases, fructanases, glucanases, xylanases and deacetylases.

In a fourth aspect of the invention, a feed additive for use in reducing post-race recovery time for Columba livia domestica is provided, the feed additive comprising a malt extract comprising one or more enzymes selected from the group consisting of amylases, maltases, cellulases, fructanases, glucanases, xylanases and deacetylases.

In a fifth aspect of the invention, a feed additive for use in improving energy utilisation originating from feed for an avian granivore is provided, the feed additive comprising a malt extract comprising one or more enzymes selected from the group consisting of amylases, maltases, cellulases, fructanases, glucanases, xylanases and deacetylases.

In a sixth aspect of the invention a feed additive for use in improving voluntary feed intake of a granivore is provided, the feed additive comprising a malt extract comprising one or more enzymes selected from the group consisting of amylases, maltases, cellulases, fructanases, glucanases, xylanases and deacetylases.

In a seventh aspect of the invention a feed additive for use in increasing egg weight of an avian granivore is provided, the feed additive comprising a malt extract comprising one or more enzymes selected from the group consisting of amylases, maltases, cellulases, fructanases, glucanases, xylanases and deacetylases.

In an eighth aspect of the invention, a feed additive for use in increasing eggshell thickness of an avian granivore is provided, the feed additive comprising a malt extract comprising one or more enzymes selected from the group consisting of amylases, maltases, cellulases, fructanases, glucanases, xylanases and deacetylases.

In a ninth aspect of the invention, a feed additive for use in improving the feed conversion ratio of an avian granivore is provided, the feed additive comprising a malt extract comprising one or more enzymes selected from the group consisting of amylases, maltases, cellulases, fructanases, glucanases, xylanases and deacetylases.

In a tenth aspect of the invention, a feed additive for use in increasing the firmness of avian granivore droppings is provided, the feed additive comprising a malt extract comprising one or more enzymes selected from the group consisting of amylases, maltases, cellulases, fructanases, glucanases, xylanases and deacetylases.

In an eleventh aspect of the invention, a feed additive for use in balancing the profile of the gut microbiome of an avian granivore is provided, the feed additive comprising a malt extract comprising one or more enzymes selected from the group consisting of amylases, maltases, cellulases, fructanases, glucanases, xylanases and deacetylases.

### Example 1 (Pigeons)

1.5 ml of EquiNectar (Tharos Limited, London, England) was diluted to 60 ml in water and fed to each of about 60 racing pigeons per day in conjunction with a normal seed based feed for a period of 3 months. The drinking troughs and surrounding area were disinfected daily.

Over the 3 month period, it was observed that the performance and recovery time of non-juvenile birds (i.e. older than about 8 weeks) improved such that they could be entered for more races with increased success. In particular, racing pigeons are normally raced twice over a three week period. However, following treatment with EquiNectar, the pigeons could be successfully raced every week. It was also observed that the percentage loss of juvenile pigeons (first about 8 weeks of life) over the same period of time of 3 months that had previously been 30-50% reduced to 0%. Furthermore, it was observed that successfully treating an E. coli outbreak only required a single dose of antibiotics rather than the two or three doses of antibiotics previously used to treat such outbreaks before treatment with EquiNectar. It was also observed that the bird population was less susceptible to viral infections following treatment with EquiNectar.

There were obvious changes in the microbiome following treatment with EquiNectar as the bird droppings, which had previously been semi-liquid and greenish in colour, became solid, white/brown in colour, and could easily be removed by hand (probably due to an increase in uric acid). Excretion of uric acid is a mechanism for excretion of nitrogen which is indicative of a healthy bird.

The foregoing observation is supported by the results of an analysis of volatile organic compounds (VOC's) contained in faecal samples of birds treated with EquiNectar with control birds not treated with EquiNectar (i.e., where the EquiNectar is replaced with water) conducted using selected ion-flow tube mass spectrometry (SIFT/MS). In brief, faecal samples of 10 non-juvenile birds treated with EquiNectar and 10 non-juvenile control birds were collected 2 months after treatment with EquiNectar commenced. The samples were stored at −80 degrees centigrade until required for testing at which time the samples were defrosted and 5 g of each sample placed in a sample bag constructed from Nalophan tubing. Each sample bag was filled with hydrocarbon free air, sealed and placed in an incubator for 45 minutes to increase compound volatilization. After incubation, the sample bags were attached to the SIFT/MS via a heated sampling capillary. Precursor ions, such as H₃O⁺, NO⁺ or O₂⁺, are generated in an air and water mixture by a microwave discharge and selected using a quadrupole mass filter and carried to the sample via helium carrier gas and react with the VOC's producing product ions which are then separated downstream quadrupole mass filter before being detected and counted by the mass spectrometer. The results are summarised in Table 1.

TABLE 1
Volatile metabolites in racing pigeon droppings (ppb)
after 2 months of treatment with EquiNectar (nd =
not detectable; each value is an average of 10 readings).

| Volatile metabolite (ppb) | Control | EquiNectar |
|---|---|---|
| NH₃ (ammonia) | 505 | 1225 |
| HCN (hydrogen cyanide) | 16 | 4 |
| CH₂O (formaldehyde) | 109 | nd |
| CH₃OH (methanol) | 295 | 235 |
| CH₃CH₂OH (ethanol) | 195 | 238 |
| CH₃CN (acetonitrile) | 110 | nd |
| CH₃CHO (acetaldehyde) | 146 | 162 |
| CH₃CH₂CH₂OH (propanol) | nd | nd |
| CH₃COCH₃ (acetone) | 33 | 80 |
| C₆H₅CH₃ (toluene) | 13 | 19 |
| CH₃COOH (acetic acid) | 138 | 96 |
| CH₃CH₂COOH (propionic acid) | 91 | 90 |
| CH3CH2CH₂COOH (butyric acid)^{a} | 96 | 768 |

^{a}Includes comparatively low levels of CH₃CH₂COOCH₃ (methyl propionate) and CH₃COOCH₂CH₃ (ethyl acetate) compared to butyric acid.

The results in Table 1 show a reduction in more toxic metabolites such as hydrogen cyanide, formaldehyde, methanol, and acetonitrile, following treatment with EquiNectar and corresponding increases in VOC's thought to be beneficial such as acetone and the short chain fatty acid butyric acid. It is thought by the inventors that the presence of toxic metabolites are a result of starch metabolism in the lower gut and a reduction in toxic metabolites allows for an increase in the gut bacteria which ultimately leads to better feed conversion and racing performance. According to Dabek et al. (‘Modulation of cellular biochemistry, epigenetics and metabolomics by ketone bodies. Implications of the ketogenic diet in the physiology of the organism and pathological states’, Nutrients, 12, 788 (2020)), ketone bodies such as beta-OH-butyrate, acetoacetic acid and acetone provide an alternative energy source to glucose and are produced by the liver from fatty acids (although not necessarily short chain fatty acids) during prolonged or intense physical activity, fasting etc. Fatty acid catabolism provides acetyl coenzyme A (acetyl CoA) then ketone bodies. Acetone is a marker of fatty acid metabolism. Garcia et al., ‘Ketone bodies are mildly elevated in subjects with Type 2 diabetes mellitus and are inversely associated with insulin resistance as measured by the lipoprotein insulin resistance index’, J. Clinical Medicine, 9, 2 Jan. 23 (2020)) concludes that concentrations of ketone bodies are inversely associated with insulin resistance. Thus, the latter results are consistent with increased utilisation of short chain fatty acid pathways which would provide more energy than glycolysis and improved glucose uptake into the muscles. This is because fatty acids have a higher carbon and hydrogen to oxygen ratio than carbohydrates. As carbon is oxidised to CO₂ and hydrogen is oxidised to H₂O, providing the supply of oxygen is not rate limiting, fats provide more energy than carbohydrates.

Also observed was a decrease in acetic acid in birds treated with EquiNectar which would be expected to lead to improvements to bird metabolism. According to Pinchasov et al. (‘Broiler chick response to anorectic agents 1. Dietary acetic and propionic acids and the digestive system’, Pharmacol. Biochem. and Behaviour, 48, 2, 371 (1994)), acetic acid supplementation to the feed of female broiler chicks resulted in a reduction in voluntary feed intake. In a study with dietary supplementation of chicken feed by 0.24% acetic acid described by Van Immerseel et al. (‘Microencapsulated short chain fatty acids in feed modify colonisation and invasion early after infection with Salmonella enteritidis in young chickens’, Poultry Science, 83, 1, 69 (2004)), it was found that there was an increase in colonisation of the chicken caeca and internal organs by Salmonella enteritidis when the birds were challenged with the bacteria. However, birds receiving propionic acid were colonized with Salmonella enteritidis to the same extent as controls. Butyric acid resulted in a significant decrease of colonization by Salmonella enteritidis in the caeca but not in the liver and spleen. Thus reducing acetic acid levels is expected to improve feeding and reduce colonisation by pathogenic bacteria.

### Example 2 (Chickens)

Free range laying birds (5) were fed with the malt-based supplement EquiNectar for two months at a level of 0.6 ml/kgbird/day. The droppings were collected before and after treatment and compared with those of 5 control birds (i.e., where the EquiNectar is replaced with water) using SIFT/MS to determine the levels of VOCs. The sampling and testing method was as described in Example 1. The results are summarised in Table 2.

TABLE 2
Volatile metabolites in chicken droppings (ppb) after 2 months of
treatment with Equinectar (each value is an average of 5 readings).

| | | | Finish/start | | | Finish/start |
|---|---|---|---|---|---|---|
| Volatile metabolite | Control | Control | value × 100 | EquiNectar | EquiNectar | value × 100 |
| (ppb) | (start) | (finish) | (control) | (start) | (finish) | (EquiNectar) |
| NH₃ (ammonia) | 37378 | 7736 | 20.69 | 46659 | 9534 | 20.45 |
| CH₂O (formaldehyde) | 10 | 69 | 690 | 16 | 5 | 31.25 ↓ |
| CH₃OH (methanol) | 2478 | 1240 | 50.04 | 2341 | 1050 | 44.85 |
| CH₃CH₂OH (ethanol) | 1091 | 3560 | 326.3 | 2312 | 3627 | 156.9 ↓ |
| CH₃CHO | 201 | 113 | 56.22 | 481 | 541 | 112.5 |
| (acetaldehyde) | | | | | | |
| CH₃COCH₃ (acetone) | 129 | 40 | 31.01 | 139 | 52 | 37.41 |
| C₄H₉OH (butanol) | 17 | 95 | 558.8 | 162 | 24 | 14.81 ↓ |
| CH₃COOH (acetic | 75 | 93 | 124.0 | 95 | 81 | 85.26 ↓ |
| acid) | | | | | | |
| C₃H₇COOH (butyric | 457 | 162 | 35.45 | 115 | 185 | 160.9 ↑ |
| acid) | | | | | | |
| CH₃CHO | 201 | 113 | 56.22 | 481 | 541 | 112.5 |
| (acetaldehyde) | | | | | | |
| CH₃SSCH₃ | 7 | 35 | 500 | 31 | 13 | 41.94 ↓ |
| (dimethyldisulphide) | | | | | | |

The microbiome of chickens changes over time and reflects diet and the environment (Khan et al., ‘The gut microbiota of laying hens and its manipulation with prebiotics and probiotics to enhance gut health and food safety’, Applied and Environmental Microbiology, 86, 13 (2020)). However, when the average values for the chickens treated with EquiNectar are compared with the controls, the results are similar to those seen for racing pigeons in Example 1, involving decreases in toxic metabolites such as formaldehyde, dimethyl disulphide, ethanol, butanol and acetic acid, with an increase in butyric acid, a main substrate for the gut colonocytes.

These changes in VOCs are due to changes in the gut microbiome. At the phylum level the gut microbiota of laying chickens is dominated by Proteobacteria, Firmicutes, Bacteroidetes, Fusobacteria and Actinobacteria, but the relative proportions depend on the age and genetic strain of the birds and on environmental factors. Samples of droppings from control and birds treated with EquiNectar as described above over a period of two months were collected and processed as before and the gut microbiome content was determined by 16S metagenomic analysis (Illumina Incorporated (CA, US) platform). The gut microbiome was profiled using error-corrected 454 pyrosequencing data from the 16S rRNA amplicons. This gave identification of the phyla, class, order, family, genus and species with estimates of the relative frequencies (percentage of total hits). These results proved to be complex.

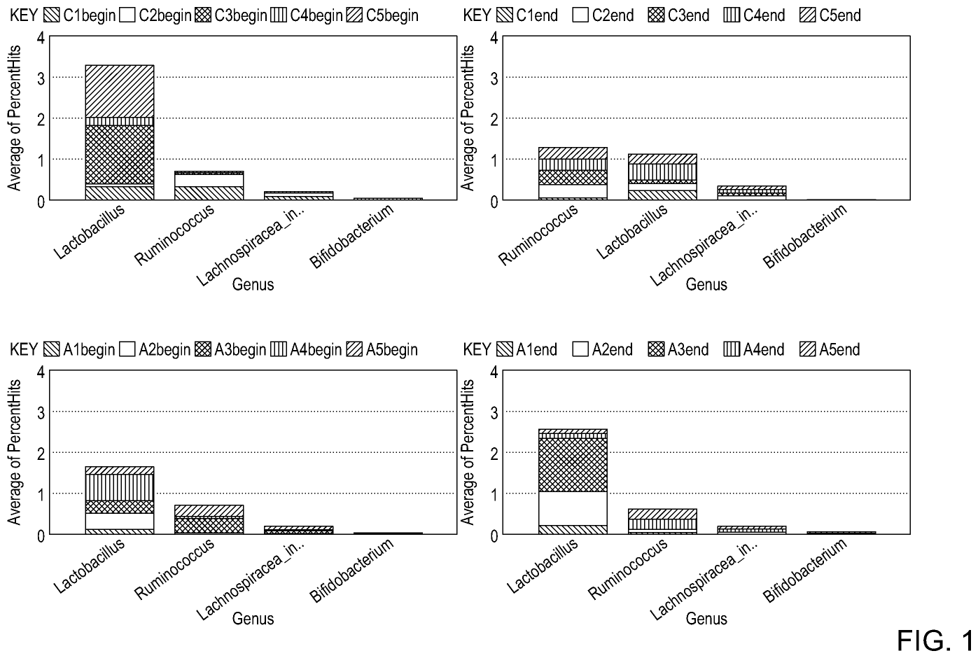

FIG. 1 shows the average percentage hits of Lactobacillus, Ruminococrus, Lachnospiracea, and Bifidobacterium for five egg laying chickens (denoted 1 to 5) before (‘begin’) and after (‘end’) 2 months of treatment with EquiNectar (denoted ‘C’) versus five egg laying control chickens where water is substituted for Equinectar (denoted ‘A’). Lactobacillus, Ruminococrus, Lachnospiracea, and Bifidobacterium are believed to be associated with improved health in chickens (‘good’ bacteria) at least in part because they all convert substrates into SCFAs (short chain fatty acids), especially butyrate (Shu et al., ‘Bamboo leaf flavone changed the community of cecum microbiota and improved the immune function in broilers’, Scientific Reports, 10, 1, 12324 (2020)). Ruminococcus species break down dietary fibre and starch and higher levels lead to improved feed conversion ratios. Lactobacillus species also produce bacteriocins which modify gut receptors against pathogenic microbes as well as improving feed efficiency (Yadav and Jha, ‘Strategies to modulate the intestinal microbiota and their effects on nutrient utilisation, performance and health of poultry’, Journal of Animal Science and Biotechnology, 10, 2 (2019)). The authors discuss the requirement for a stable and balanced microbiome profile that is essential for a healthy host. FIG. 1 shows that in birds treated with EquiNectar, Ruminococcus and Lachnospiraceae genera are increased and the levels of Lactobacillus falls in some birds leading to a more balanced profile overall. In the control birds, the opposite picture is observed.

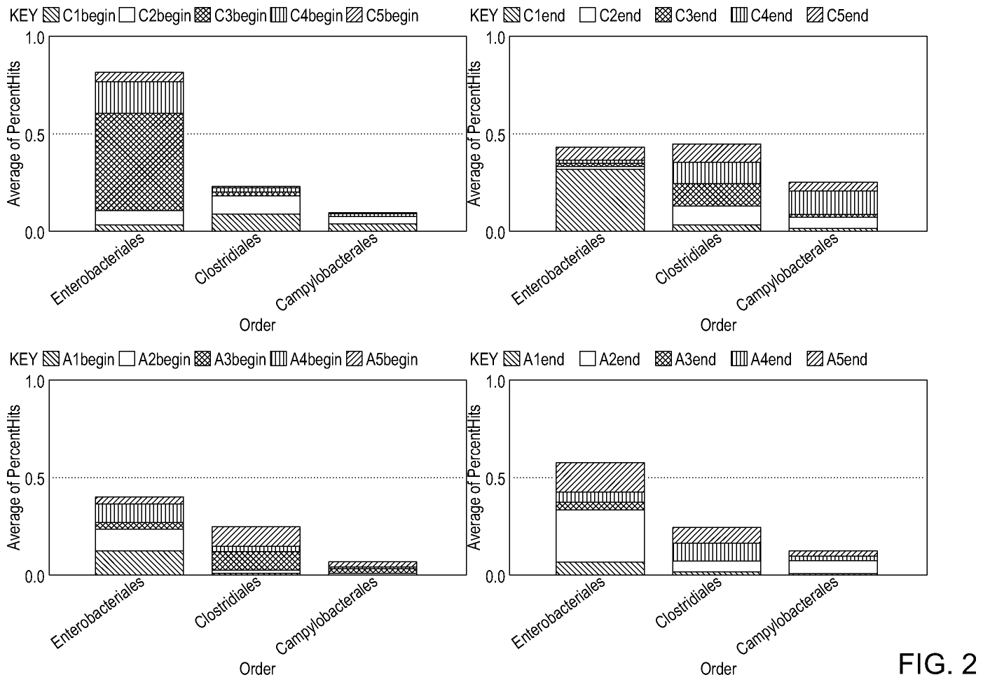

FIG. 2 shows the average percentage hits of Enterobacteriales, Clostridiales, and Campylobacterales for five egg laying chickens (denoted 1 to 5) before (‘begin’) and after (‘end’) 2 months of treatment with EquiNectar (denoted ‘C’) versus five egg laying control chickens where the water is substituted for Equinectar (denoted ‘A’). Enterobacteriales, Clostridiales, and Campylobacterales are considered ‘toxic’ bacteria associated negatively with chickens health. Here, the levels of Enterobacteriales are reduced in supplemented birds, although they increased in the controls. Enterobacteriaceae are involved with the proliferation of antimicrobial resistance as well as being potential producers of gut toxins (Gupta et al., ‘Longitudinal study on the effects of growth-promoting and therapeutic antibiotics on the dynamics of chicken cloacal and litter microbiomes and resistomes’, Microbiome, 9, 1, 178 (2021)).

It is clear that EquiNectar supplementation changes the chicken microbiome, altering it in a positive way. The weights of the birds, together with egg weights, were therefore determined at the start and finish of the aforementioned trial. Egg weight and shell thickness were measured using a micrometer over a period of 4 weeks (start and end of trial). Bird weight was measured over a period of 2 months, whereas the consistency of droppings and behavioural changes was measured or observed over a period of 1 week. The results are shown in Table 3.

TABLE 3
Weights of chickens and eggs before and after supplementation
with Equinectar, and eggshell thickness.

| | Egg | Egg | Shell | Shell | Chicken | Chicken | | |
|---|---|---|---|---|---|---|---|---|
| | weight | weight | thickness | thickness | weight | weight | Consistency | |
| | before | after | before | after | before | after | of | Behavioural |
| Bird | (g) | (g) | (mm) | (mm) | (g) | (g) | droppings | changes |
| EquiNectar 1 | 6.0 | 6.9 | 0.4 | 0.8 | 1760 | 1810 | Much | Calm |
| | | | | | | | firmer | |
| EquiNectar 2 | 6.0 | 6.9 | 0.4 | 0.8 | 1680 | 1760 | Much | Calm |
| | | | | | | | firmer | |
| EquiNectar 3 | 5.9 | 6.9 | 0.4 | 0.9 | 1840 | 1905 | Much | Calm |
| | | | | | | | firmer | |
| EquiNectar 4 | 6.0 | 6.9 | 0.4 | 0.9 | 1725 | 1850 | Much | Calm |
| | | | | | | | firmer | |
| EquiNectar 5 | 6.0 | 6.8 | 0.4 | 0.9 | 1650 | 1740 | Much | Calm |
| | | | | | | | firmer | |
| Control 1 | 5.8 | 5.8 | 0.4 | 0.4 | 1740 | 1745 | Soft | Agitated |
| Control 2 | 5.8 | 5.8 | 0.4 | 0.4 | 1725 | 1745 | Soft | Agitated |
| Control 3 | 5.8 | 5.8 | 0.4 | 0.4 | 1700 | 1710 | Soft | Agitated |
| Control 4 | 5.9 | 5.9 | 0.5 | 0.5 | 1695 | 1705 | Soft | Agitated |
| Control 5 | 5.9 | 5.9 | 0.4 | 0.4 | 1660 | 1660 | Soft | Agitated |

During the trial period the weights of the control group did not differ significantly when start and finish values were determined. (Mean 1704 g v. 1731 g, p=0.24). However, the EquiNectar group weights rose significantly (Mean 1713 g v. 1813 g, p=0.0091). The control hens' mean egg weight was 5.84 g which remained unchanged at the end of the study whilst the EquiNectar-supplemented group mean egg weight rose from 5.96 g to 6.8 g (p<0.00001). The control eggshell thickness remained unchanged (Mean 0.42 v. 0.44 mm) while in the supplemented group, the eggshell thickness increased from 0.4 mm to 0.86 mm (p<0.00001).

The feed conversion ratio therefore increased on supplementation with EquiNectar while the metagenomic analysis and observation of the increased consistency of the droppings are consistent with positive changes in the microbiome. The change in the consistency of avian droppings is of commercial value in maintaining/improving hygiene. Further, the egg weights and shell thickness increased on supplementation with EquiNectar. As all hens had crushed oyster shell (largely calcium carbonate) available ad lib as a calcium supply, the increased eggshell thickness must reflect improved calcium absorption/utilisation on EquiNectar dosage.

1. A method of avian granivore treatment or prevention, the method comprising administering a feed additive to an avian granivore, the feed additive comprising:
a malt extract comprising one or more enzymes selected from the group consisting of amylases, maltases, cellulases, fructanases, glucanases, xylanases and deacetylases.

2-10. (canceled)

11. The method of avian granivore treatment or prevention according to claim 26, wherein the one or more pathogenic bacteria are gastrointestinal pathogenic bacteria.

12. The method of avian granivore treatment or prevention according to claim 26, wherein the one or more pathogenic bacteria is selected from the group consisting of Escherichia coli, Salmonella enteritidis, Campylobacter spp, and Staphylococcus spp.

13. The method of avian granivore treatment or prevention according to claim 1, wherein the malt extract additionally comprises one or more proteinases and/or lipases.

14. The method of avian granivore treatment or prevention according to claim 1, wherein the malt extract additionally comprises one or more water soluble sugars selected from the group consisting of maltose, maltotriose, and maltose polymers.

15. The method of avian granivore treatment or prevention according to claim 1, wherein the malt extract is based on one of the seeds selected from the group consisting of barley, wheat, triticale, sorghum, maize, buck wheat, rice and a mixture thereof.

16. The method of avian granivore treatment or prevention according to claim 1, wherein the daily dosage of the malt extract is 0.3-30, 0.4-20, 0.5-10, 0.7-5, 1-3 g per 1000 g granivore body weight.

17. The method of avian granivore treatment or prevention according to claim 1, wherein the diastatic power of the malt extract is above 35, 40, 45, 50, 55, 60, 65, 70, 75 or 80 degrees Lintner.

18. The method of avian granivore treatment or prevention according to claim 1, wherein the feed additive additionally comprises one or more medium chain triglycerides.

19. The method of avian granivore treatment or prevention according to claim 18, wherein the feed additive comprises 1-5, 1-10 or 1-15 w/w one or more medium chain triglycerides.

20. The method of avian granivore treatment or prevention according to claim 18, wherein the medium chain triglyceride is coconut oil.

21. The method of avian granivore treatment or prevention according to claim 1, wherein the feed additive additionally comprises an effective amount of one or more water soluble organic acid or salt thereof.

22. The method of avian granivore treatment or prevention according to claim 21, wherein the water soluble organic acid is selected from the group consisting of caprylic acid, sorbic acid, caproic acid, benzoic acid, ascorbic acid, propionic acid, acetic acid, formic acid, fumaric acid, and tartaric acid.

23. The method of avian granivore treatment or prevention according to claim 21, wherein the feed additive comprises 0.001-5, 0.01-3, 0.05-2, 0.1-1.5, 0.5-1.25, 0.75-1.2% w/w of one or more water soluble organic acid or salt thereof.

24. The method of avian granivore treatment or prevention according to claim 1, further comprising administering granivore feed.

25. The method of avian granivore treatment or prevention according to claim 26, wherein the avian granivore is Columba livia domestica or Gallus gallus domestica.

26. The method of avian granivore treatment or prevention of claim 1, wherein the method is carried on out an avian granivore suffering from one or more pathogenic bacteria.

27. The method of avian granivore treatment or prevention of claim 1, wherein the method further comprises administration of one or more antibiotics to the avian granivore, and wherein the avian granivore is suffering from one or more pathogenic bacteria.

28. The method of avian granivore treatment or prevention of claim 1, wherein the method is carried out periodically over time to reduce the incidence of viral infections in the avian granivore.

29. The method of avian granivore treatment or prevention of claim 1, wherein the method further comprises decreasing the amount of feed provided to the avian granivore.

30. The method of avian granivore treatment or prevention of claim 1, wherein the method is carried on out an avian granivore suffering from low voluntary food intake.

31. The method of avian granivore treatment or prevention of claim 1, wherein the method is carried on out an avian granivore suffering from low egg weight.

32. The method of avian granivore treatment or prevention of claim 1, wherein the method is carried on out an avian granivore suffering from low egg shell thickness.

33. The method of avian granivore treatment or prevention of claim 1, wherein the method is carried on out an avian granivore suffering from low feed conversion ratio.

34. The method of avian granivore treatment or prevention of claim 1, wherein the method is carried on out an avian granivore suffering from soft droppings.

35. The method of avian granivore treatment or prevention of claim 1, wherein the method is carried on out an avian granivore following a race to improve recovery time.
